(19) United States
(12) Patent Application Publication
(10) Pub. No.: US 2002/0120102 A1
(43) Pub. Date: Aug. 29, 2002

(54) Use of dendroaspin as a scaffold for non-dendroaspin domains

(76) Inventors: Xinjie Lu, London (GB); Vijay Vir Kakkar, London (GB)

Correspondence Address: FLEHR HOHBACH TEST ALBRITTON & HERBERT LLP, Suite 3400, Four Embarcadero Center, San Francisco, CA, 94111-4187, US

(21) Appl. No.: 09/779,054

(22) Filed: Feb. 5, 2001

(30) Foreign Application Priority Data
Feb. 5, 2000 (GB) 0002625.2

(51) Int. Cl. C07K 14/435; C07H 21/04; C12P 21/02; C12N 5/06

(52) U.S. Cl. 530/350; 536/23.5; 435/320.1; 435/69.1; 435/325

(57) ABSTRACT

The use of dendroaspin as a scaffold for one or more non-wild-type dendroaspin domains, the dendroaspin scaffold being modified in that the native RGD motif has been deleted or has been replaced by (i) an amino acid sequence having no integrin-binding activity or (ii) an integrin-binding amino acid sequence other than RGD which contains aspartic acid (D) or glutamic acid (E).

### FIELD OF THE INVENTION

The present invention relates to molecules comprising modified dendroaspin scaffolds and in particular to the use of dendroaspin as a scaffold for non-dendroaspin amino acid sequences.

### BACKGROUND OF THE INVENTION

The role of blood coagulation is to provide an insoluble fibrin matrix for consolidation and stabilisation of a haemostatic plug (blood clot). Formation of a cross-linked fibrin clot results from a series of biochemical interactions involving a range of plasma proteins.

Acute vascular diseases, such as myocardial infarction, stroke, pulmonary embolism, deep vein thrombosis and peripheral arterial occlusion are caused by either partial or total occlusion of a blood vessel by a blood clot.

The formation of a blood clot within a blood vessel is termed thrombosis and is dependent upon platelet aggregation. In the context of blood vessel injury (such as that which might arise in surgical procedures), the interaction of blood platelets with the endothelial surface of injured blood vessels and with other platelets is a major factor in the course of development of clots or thrombi.

Platelet aggregation is dependent upon the binding of fibrinogen and other serum proteins to the glycoprotein receptor IIb/IIIa complex on the platelet plasma membrane. Glycoprotein GP IIb/IIIa is a member of a large family of cell adhesion receptors known as integrins, many of which are known to recognise an Arg-Gly-Asp (RGD) tripeptide recognition sequence. Integrins are a family of cell surface receptors that mediate adhesion of cells to each other or to extracellular matrix substrate. Kieffer et al (1990) Ann. Rev. Cell Biol. 6, 329-357; Hynes (1992) Cell 69, 11-25; McEver (1992) Curr. Opin. Cell Biol. 4, 840-849; Smyth et al. (1993) Blood 81, 2827-2843; Giancotti et al. (1994) Biochim. Biophys. Acta. 1198, 47-64. They are composed of non-covalently associated α and β transmembrane subunits selected from among 16α and 8β subunits that heterodimerise to produce 20 receptors. Pierschbacher et al. (1984) Nature 309, 30-33. Among the integrins, the platelet membrane α_{IIb}β₃ is the best characterised. McEver, supra; Giancotti et al., supra. Upon cell activation, the α_{IIb}β₃ integrin binds several glycoproteins, predominantly through the Arg-Gly-Asp (RGD) tripeptide sequence (Pierschbacher et al., supra; Plow et al. (1987) Blood 70, 110-115; Pytela et al. (1986) Science 231, 1559-1162) present in fibrinogen (Nachunan et al. (1992) J. Clin. Invest. 69, 263-269), fibronectin (Gardner et al. (1985). Cell 42, 438-449), von Willebrand factor (Ruggeri et al. (1983) J. Clin. Invest. 72, 1-12), vitronectin (Pytela et al. (1985) Proc. Natl. Acad, Sci. USA 82, 5766-5770) and thrombospondin (Karczewski et al. (1989) J. Biol. Chem. 264, 21322-21326). The nature of the interactions between these glycoprotein ligands and their integrin receptors is known to be complex with conformation changes occurring in both the receptor (Sims et al. (1991) J. Biol. Chem. 266, 7345-7352) and the ligand (Ugarova et al. (1995) Thrombosis and Haemostasis 74, 253-257).

The practical effect of such interactions can be illustrated by considering the treatment of localised narrowing of an artery caused by atherosclerosis. This is a condition which can usually be remedied surgically by the technique of balloon angioplasty. The procedure is invasive and causes some tissue damage to the arterial wall which can result in thrombus formation. Extracellular proteins such as fibronectin in the arterial wall become exposed to blood in the artery. Platelets bind to the RGD motif of fibronectin via integrin receptors which in turn leads to platelet aggregation and the start of the cascade of clotting reactions. An agent which specifically inhibits platelet aggregation at the sites of damage and which also inhibits clotting at these sites is required. The agent should be non-toxic and free of undesirable side effects such as a risk of generalised bleeding.

Various agents for preventing formation of blood clots are now available, such as aspirin, dipyridamole and filopidine. These products generally inhibit platelet activation and aggregation, or delay the process of blood coagulation, but they have the potential side effect of causing prolonged bleeding. Moreover, the effect of such products can be reversed only by new platelets being formed or provided.

Therefore, the development of antagonists towards selected cell adhesion events would be of significant clinical utility in the treatment of thrombosis and atherosclerosis. A key cell adhesion mechanism common to a number of integrin-ligand interactions involves the recognition of aspartic acid (D)-containing sequences or motifs identified by the use of inhibitory synthetic peptide analogues including RGD, KGD, LDV, KQAGDV (SEQ ID NO: 15). However, these peptides are limited by low potency and specificity. In this regard, a major breakthrough has been the discovery of a family of small, RGD-containing proteins derived from snake venoms termed disintegrins.

Scarborough et al (1991) J. Biol. Chem. 266, 9359-9362, have reported a naturally occurring KGD-containing snake protein isolated from the venom of Sistrurus M Barbouri termed barbourin showing a GPIIb-IIIa specific integrin antagonist activity.

Recently, many proteins from a variety of snake venoms have been identified as potent inhibitors of platelet aggregation and integrin dependent cell adhesion. The majority of these proteins which belong to the disintegrin family share a high level of sequence homology, are small (4-8 kDa), cysteine rich and contain the sequence RGD (Gould et al. (1990) Proc. Soc. Exp. Bio. Med. 195, 168-171) or KGD (Scarborough et al., supra) In addition to the disintegrin family, a number of non-disintegrin RGD proteins of similar inhibitory potency, high degree of disulphide bonding and small size, have been isolated from both the venoms of the Elapidae family of snakes (McDowell et al. (1992) Biochemistry 31, 4766-4772; Williams et al. (1992) Biochem. Soc. Trans. 21, 73S) and leech homogenates (Knapp et al. (1992) J. Biol. Chem. 267, 24230-24234). All of these proteins are approximately 1000 times more potent inhibitors of the interactions of glycoprotein ligands with the integrin receptors than simple linear RGD peptides—a feature that is attributed to the optimally favourable conformation of the RGD motif held within the protein scaffold. The NMR structures of several inhibitors including kistrin (Adler et al. (1991) Science 253, 445-448; Adler & Wagner (1992) Biochemistry 31, 1031-1039; Adler et al. (1993) Biochemistry 32, 282-289), flavoridin (Senn et al. (1993) J. Mol. Biol. 234, 907-925), echistatin (Saudek et al. (1991a) Biochemistry 30, 7369-7372; Saudek et al. (1991b) Eur. J. Biochem. 202, 329-338; Cooke et al. (1991) Eur. J. Biochem. 202, 323-328; Cooke et al. (1992) Protein Eng. 5, 473-477), albolabrin (Jaseja et al. (1993) Eur. J. Biochem. 218, 853-860), decorsin (Krezel et al. (1994) Science 264, 1944-1947) and dendroaspin (Jaseja et al. (1994) Eur. J. Biochem. 226, 861-868; Sutcliffe et al. (1994) Nature Structure Biology 1, 802-807) have been reported and the only common structural feature elucidated so far is the positioning of the RGD motif at the end of a solvent exposed loop, a characteristic that is of prime importance to their inhibitory action.

Dendroaspin, therefore, is a natural variant of the short neurotoxin family, but contains the adhesive tripeptide Arg-Gly-Asp (RGD) and functions as a potent antagonist of integrin-mediated cell adhesive interactions. Dendroaspin was originally isolated from the venom of the Elapidae snake Dendroaspis jamesonii (Jameson's mamba) as a potent inhibitor of platelet aggregation and integrin mediated platelet adhesion. The activity of dendroaspin is due to an RGD motif contained within a solvent-exposed loop. International patent application WO 98/42834 describes amongst other things bi- or multi-functional molecules based on a dendroaspin scaffold, in which, in addition to integrin-binding function, a second function is achieved by grafting a domain of another protein into a dendroaspin scaffold. WO 98/42834 and its entire content is included herein by reference.

As is described in WO 98/42834, the dendroaspin molecule has 59 amino acid residues and comprises 3 loops. Loop I comprises amino residues 4-16, loop II residues 23-36 and loop III residues 40-50; it is loop III which contains the RGD motif in wild-type dendroaspin. The RGD domain forms residues 43-45.

### SUMMARY OF THE INVENTION

The following abbreviations are used in this specification:

Hydrophobic amino acids

A=Ala=alanine

V=Val=valine

I=Ile=isoleucine

L=Leu=leucine

M=Met=methionine

F=Phe=phenylalanine

P=Pro=proline

W=Trp=tryptophan

Polar (uncharged) amino acids

N=Asn=asparagine

C=Cys=cysteine

Q=Gln=glutamine

G=Gly=glycine

S=Ser=serine

T=Thr=threonine

Y=Tyr=tyrosine

Positively charged amino acids

R=Arg=arginine

H=His=histidine

K=Lys=lysine

Negatively charged amino acids

D=Asp=aspartic acid

E=Glu=glutamic acid

The present invention relates to products comprising a modified dendroaspin scaffold. The dendroaspin scaffold has been found to form a stable framework for non-dendroaspin sequences and to be useful for this purpose irrespective of whether the modified scaffold retains the RGD sequence or, indeed, any integrin-binding activity at all. Molecules comprising a dendroaspin scaffold in which the RGD motif has been replaced by another integrin-binding motif but into which no further functional sequence has been introduced are useful scientific tools, for example for the study of receptor interactions, as can be polypeptides comprising a dendroaspin scaffold in which the RGD motif has been deleted or replaced by a non-integrin-binding motif.

### BRIEF DESCRIPTION OF THE DRAWING

FIG. 1 comprises alignments of modified dendroaspins (SEQ ID NOS: 1-14) where the inserted amino acid sequences are listed beneath the amino acid sequence of dendroaspin.

### DETAILED DESCRIPTION OF THE INVENTION

The invention relates preferably to the use of dendroaspin as a scaffold for one or more non-wild-type dendroaspin domains, the dendroaspin scaffold being modified in that the native RGD motif has been deleted or has been replaced by (i) an amino acid sequence having no integrin-binding activity or (ii) an integrin-binding amino acid sequence other than RGD which contains aspartic acid (D) or glutamic acid (E).

The present invention further provides in one aspect a product comprising a dendroaspin scaffold in which the native RGD motif has been deleted or has been replaced by a replacement amino acid sequence. In one class of products, the replacement amino acid sequence is an amino acid sequence having no integrin-binding activity. In another class of products, the replacement amino acid sequence is an integrin-binding amino acid sequence and comprising a tripeptide sequence other than RGD containing D or E adjacent to G or to a hydrophobic amino acid.

In preferred products the tripeptide sequence is of the formula

B-J-Z

wherein

(I) J-Z is GD or GE and B is R, K, Q, A, H, N, A, V, I, L, M, F, P or W;

(II) B-J is DG or EG and Z is any amino acid; or

(III) J is D or E and B and Z are each independently selected from A, V, I, L, M, F, P or W.

Preferably J-Z is GD and, in the products in which J-Z is GD or GE, B is preferably R, K, Q, A, H or N and more preferably is R, K, Q or A (but is not R when J-Z is GD).

A preferred class of products (I) comprises those in which B-J-Z is bonded at its C-terminal end to M, W, N or V. Preferably the M, W, N or V residue is followed by the P which is at position 47 of wild type dendroaspin or by an A residue substituted therefor.

Another preferred class of products (I) comprises those in which the integrin-binding amino acid sequence is preceded by the P which is at position 42 of wild type dendroaspin or by an A residue substituted therefor.

In some preferred products (I), especially those in which J-Z is GD, B is A, V, I, M, F, P, W and more preferably is L or V. The most preferred products of this type are those in which B is L and is preceded by M.

Preferred products (II) include those in which B-J is DG and/or Z is E, R or P, and especially in which Z is followed by the P which is at position 47 of wild type dendroaspin or by an A inserted before the wild type position 47 P.

A preferred class of products (III) comprises those in which J is D and, more particularly, B-J-Z is LDV. B-J-Z is preferably preceded by an I residue.

If the RGD motif is replaced by a non-integrin-binding sequence, the replacement sequence may in principle be any sequence which permits a dendroaspin-like configuration to remain, for example it may be a non-dendroaspin domain as described in more detail later in this specification. Of course, the modified dendroaspins of the invention will often have a configuration which differs somewhat from that of wild-type dendroaspin but do normally have a three-loop structure. Preferably, the RGD-replacement associates with a receptor pocket or another pocket, since loop III is favourable for pocket-binding sequences; such sequences include the thrombin-binding sequence GPRP (SEQ ID NO: 16) and the collagen α₂β₁-binding sequence DGE.

In addition to having a deleted or replaced RGD motif, the polypeptides of the invention usually comprise in the dendroaspin scaffold at least one non-wild-type dendroaspin domain elsewhere than the native RGD site. The at least one non-wild-type dendroaspin domain usually comprises at least one non-dendroaspin sequence which confers functionality on the polypeptide.

One class of polypeptides have an integrin-binding activity which, when administered in vivo, results in the binding of the molecules to platelets thereby inhibiting the aggregation of the platelets, at sites of injury. In these polypeptides, the RGD motif has been replaced by another platelet-binding sequence, especially KGD. In addition to containing an integrin-binding domain, the polypeptides of this class preferably contain another non-wild-type dendroaspin domain which provides secondary, optionally further, functionality e.g. antithrombotic action, inhibition of cell migration and/or proliferation, or regulation of signal transduction. Molecules of this class of the invention are therefore bi- or multi-functional in their activities, and preferably are bi- or multi-functional in their activities against blood coagulation, particularly thrombus formation and arterial/venous wall thickening at the sites of injury. Polypeptides of the invention may have activity against leukocyte recruitment, immune system activation, tissue fibrosis or tumorigenesis.

The polypeptide may comprise at least two non-wild-type dendroaspin domains, said domains optionally having the same sequence.

Optionally, the molecules of the invention include a dendroaspin scaffold containing a non-wild-type dendroaspin domain which comprises two or more amino acid sequence portions separated by at least one amino acid residue of dendroaspin. The two or more sequence portions may be transposed with respect to one another and to the linear order of amino acids in the native non-dendroaspin amino acid sequence. In other words, the native order of the two or more amino acid sequence portions may be altered without the actual sequence of each portion necessarily being altered (although the sequence of at least one portion may be modified).

Most polypeptides of the invention contain a domain not found in wild-type dendroaspin, i.e. a non-wild-type dendroaspin domain. The non-wild-type domain usually confers a function on the molecule, although in the case of molecules prepared for the purpose of scientific studies the domain may not always confer a function. The functionality conferred by the non-wild-type domain is not critical to the invention and in principle may be any function capable of being conferred by an amino acid sequence which can be incorporated in the dendroaspin scaffold. For example, and especially when it has the RGD motif replaced by a D or E-containing motif conferring platelet-binding activity, the polypeptide may contain a non-wild-type dendroaspin domain comprising a sequence conferring platelet derived growth factor (PDGF) activity, glycoprotein IBα activity, hirudin activity, thrombomodulin activity, vascular epidermal growth factor activity, transforming growth factor-β1 activity, basic fibroblast growth factor activity, angiotensin II activity, factor VIII activity, tissue factor pathway inhibitor (TFPI) von Willebrand factor activity, tick anticoagulant protein (TAP) activity or nematode anticoagulant protein (NAP) activity. The non-wild-type dendroaspin domain typically comprises a sequence derived from platelet derived growth factor (PDGF), glycoprotein IBα, hirudin, thrombomodulin, vascular epidermal growth factor, transforming growth factor-β1, basic fibroblast growth factor, angiotensin II, factor VIII, tissue factor pathway inhibitor (TFPI), von Willebrand factor, TAP or NAP, or a functional sequence having homology to at least part of such sequence. Such functional sequences may share about 50% amino acid sequence homology, preferably about 65%, more preferably about 75% and even more preferably about 85% homology with dendroaspin.

In this way the molecules of the invention may be rendered multifunctional so that they are active against, for example, platelet aggregation and another component in the clotting cascade (e.g. thrombin activity), or the intracellular signalling cascade (e.g. growth factor). The bi- or multi-functional dendroaspins of the invention may be engineered to contain a said non-wild-type domain having integrin-binding activity in addition to an integrin-binding RGD replacement (X-Y-Z), thereby providing a dendroaspin based molecule with augmented integrin-binding activity. The invention includes of course dendroaspin-based molecules which contain no integrin-binding function and molecules with no anti-coagulant function.

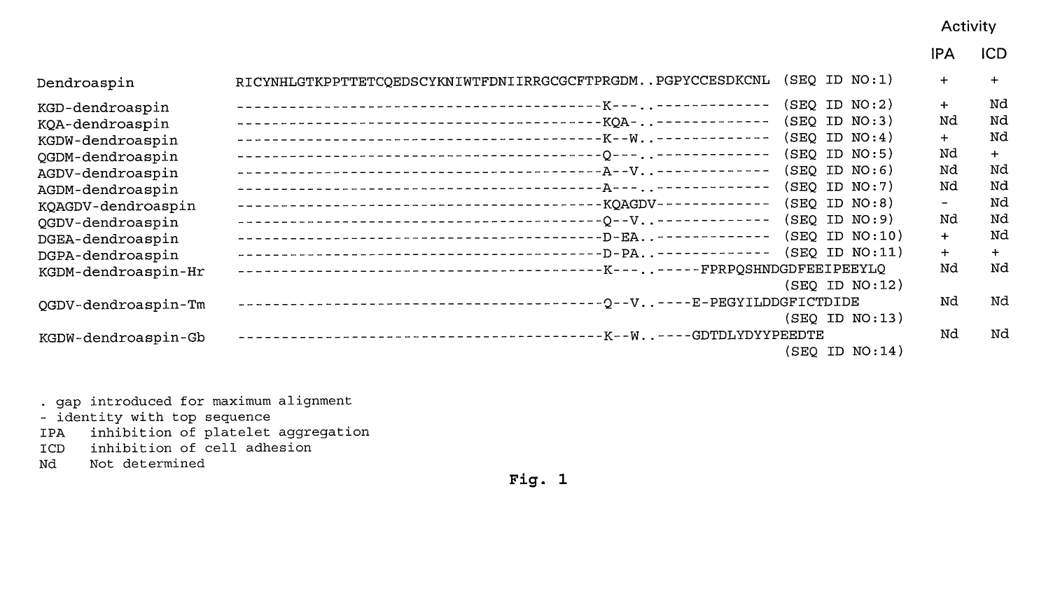

The polypeptide of the invention preferably comprises an amino acid sequence as shown in FIG. 1. Excluding said further amino acid sequence, the dendroaspin scaffolds of the invention may be molecules homologous to wild-type dendroaspin which may share about 50% amino acid sequence homology, preferably about 65%, more preferably about 75% and even more preferably about 85% homology with dendroaspin.

The polypeptides of the invention may comprise a greater or lesser number of amino acid residues compared to the 59 amino acids of dendroaspin. For example, the molecules of the invention may comprise a number of amino acid residues in the range 45 to 159, preferably about 49 to 89, more preferably about 53 to 69, even more preferably about 57 to 61. However, the inserted foreign sequences of many polypeptides of the invention replace native sequences of the same length, i.e. the one or more non-wild-type domains are the same size as the native domains they replace; if the RGD motif is replaced by a tripeptide sequence (e.g. KGD) such polypeptides will of course have 59 amino acid residues.

Preferred polypeptides comprise an amino acid sequence as shown in FIG. 1.

In one class of polypeptides, said non-wild-type domain(s) is/are incorporated into (a) loop I and/or loop II; (b) loop I and/or loop III; (c) loop II and/or loop III; or loop I, loop II and loop III of the dendroaspin scaffold. In another class, however, the polypeptides comprise a non-wild-type domain extending into or substituting regions external to the loops, i.e. residues 1-3, 17-22 and 37-39 such that residues of the non-loop regions are augmented or substituted for those of the further amino acid sequence or sequences being inserted (the non-wild-type domain or domains). If the non-wild-type domain is incorporated into a loop, it is preferably incorporated into either loop I or loop II, leaving loop III unaltered.

A preferred location for the foreign further sequence is at a site in the dendroaspin scaffold between amino acid residues: 4-16, 18-21 or 23-36, or at a site forming the C-terminus of the polypeptide, e.g. at the end of the dendroaspin scaffold after residue 50, e.g. after one of residues 52 to 59. Although foreign sequences which form the C-terminus may be inserted in their entirety after residue 50, they may alternatively commence in or before loop III, e.g. at residue 37, 38, 39, 40 or 41 or later (e.g. at residue 47).

Each inserted non-wild-type domain or portion of a non-wild-type domain is preferably an amino acid sequence having from 3 to 40 amino acid residues, more preferably 3-16, even more preferably 3-14 amino acid residues. The start of the inserted further amino acid sequence (non-wild-type domain) may be at any one of amino acid residues 1-57 of the dendroaspin scaffold. The end of the inserted amino acid sequence may be at any one of the amino acid residues 3-59 of the dendroaspin scaffold, or the inserted sequence may extend beyond the position of residue 59.

When two non-wild-type domains are inserted into the dendroaspin scaffold then the linear distance between these is preferably from 1-35 amino acids, more preferably 1-14 amino acids. When more than two non-wild-type domains are inserted then there is preferably at least one native dendroaspin amino acid residue separating each further amino acid sequence.

Loop III may be modified by insertion, deletion or substitution of any one or more amino acid residues, preferably a maximum of 8 or a minimum of 1 amino acids can be modified within loop III of dendroaspin, e.g. 1, 2, 3 or 4.

An integrin-binding sequence (e.g. KGD or RGD motif) may be introduced into the dendroaspin scaffold at a place other than the wild-type RGD domain, preferably into loop I or loop II.

The molecules of the invention in which RGD is replaced may comprise a loop III having an amino acid sequence flanking the RGD site modified from that flanking RGD in wild-type dendroaspin, for example modified as shown in FIG. 3B of WO 98/42834. An advantage of modifying the flanking region is that the activity of the B-J-Z sequence (e.g. integrin-binding activity) may be enhanced or become more specific for certain glycoprotein ligands. Also, if one or more of the “foreign” further amino acid sequences grafted into the dendroaspin scaffold has steric effects on a replacement amino acid sequence for RGD then loop III around the RGD domain (occupied by B-J-Z) can be modified to overcome any steric hindrance thereby restoring, perhaps enhancing, functionality at the RGD domain.

Especially if the replacement amino acid sequence has more than 3 residues, amino acids flanking the RGD site may be deleted, for example so that the number of amino acid residues in loop III remains as 13.

Loop I or loop II may be modified by insertion, deletion or substitution of one or more amino acid residues. Any suitable number of amino acids can be inserted into the dendroaspin scaffold to give, for example, a desired bi- or multi-functional activity although a number of residues in the range 14 to 36 are preferred for insertion at one or more sites in the dendroaspin scaffold.

Modification of the dendroaspin loops may become necessary if a “foreign” further amino acid sequence grafted into the dendroaspin scaffold has a steric hindrance effect either on another grafted domain or on the loop III. Computer assisted molecular modelling using Insight II software (Molecular Simulations Inc) can be used to predict the structure of the “loop grafted” dendroaspins of this invention. In instances where steric effects between the loops may serve to cause loss of functionality, these effects can be “designed out” by modifying appropriate parts of the dendroaspin molecule in an appropriate way. Sometimes this may involve inserting a number of suitable amino acid residues to extend one or more of the loop structures.

Preferred modification includes the insertion of polyglycine into the loop or loops of the dendroaspin scaffold in order to extend them. Other modifications comprising repeat units of an amino acid residue or number of residues can be used. Computer modelling studies can be used to design the loop modifications needed in order to extend the loops of dendroaspin.

In the design of a bi-functional or multi-functional molecule in accordance with the invention, “fine tuning” of activity, stability or other desired biological or biochemical characteristic may be achieved by altering individual selected amino acid residues by way of substitution or deletion. Modification by an insertion of an amino acid residue or residues at a selected location is also within the scope of this “fine tuning” aspect of the invention. The site-directed mutagenesis techniques available for altering an amino acid sequence at a particular site in the molecule will be well known to a person skilled in the art.

Preparation

The polypeptides of the invention may be made by construction of appropriate expression vectors, e.g. polynucleotides comprising a coding sequence operatively linked to a promoter.

The skilled person can readily construct a variety of clones containing functional nucleic acids. Cloning methodologies to accomplish these ends, and sequencing methods to verify the sequences of nucleic acids, are well known in the art. Examples of appropriate cloning and sequencing techniques, and instructions sufficient to direct persons of skill through many cloning exercises are found in Sambrook, et al., Molecular Cloning: A Laboratory Manual (2nd Ed., Vols. 1-3, Cold Spring Harbor Laboratory (1989)), Methods in Enzymology, Vol. 152: Guide to Molecular Cloning Techniques (Berger and Kimmel (eds.), San Diego: Academic Press, Inc. (1987)), or Current Protocols in Molecular Biology, (Ausubel, et al. (eds.), Greene Publishing and Wiley-Interscience, New York (1987).

Product information from manufacturers of biological reagents and experimental equipment also provide information useful in known biological methods. Such manufacturers include the SIGMA chemical company (Saint Louis, Mo.), R & D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill.

Polynucleotides containing a desired gene can be prepared by any suitable method including, for example, cloning and restriction of appropriate sequences as discussed supra, or by direct chemical synthesis by methods such as the phosphotriester method of Narang et al. Meth. Enzymol. 68: 90-99 (1979); the phosphodiester method of Brown et al., Meth. Enzymol. 68: 109-151 (1979); the diethylphosphoramidite method of Beaucage et al., Tetra. Lett., 22: 1859-1862 (1981); the solid phase phosphoramidite triester method described by Beaucage and Caruthers (1981), Tetrahedron Letts., 22(20):1859-1862, e.g., using an automated synthesizer, e.g., as described in Needham-VanDevanter et al. (1984) Nucleic Acids Res., 12:6159-6168; and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

Nucleic acids may be modified by site-directed mutagenesis, as is well known in the art. Native and other nucleic acids can be amplified by in vitro methods. Amplification methods include the polymerase chain reaction (PCR), the ligase chain reaction (LCR), the transcription-based amplification system (TAS), the self-sustained sequence replication system (SSR). A wide variety of cloning methods, host cells, and in vitro amplification methodologies are well-known to persons of skill.

As described in WO 98/42834, the wild type dendroaspin gene may be successfully inserted into a plasmid pGEX-3X (FIG. 2 of WO 98/42834) and expressed according to the method of Lu et al. (1996) J. Biol Chem 271: 289-295. Starting with the wild type gene for dendroaspin, variants of the dendroaspin gene for expressing polypeptides of the invention may then be engineered using recombinant DNA technology. For the longer insertion variants, oligonucleotides which encode the non-dendroaspin or heterologous amino acids may simply be inserted directly into suitably restriction digested wild type dendroaspin gene and then ligated. For minor changes such as modification of a few amino acid residues including the insertion, substitution or deletion, site directed mutagenesis may be used, for example using the Transformer™ Site-Directed Mutagenesis kit from Clontech Laboratories in accordance with the manufacturer's instructions.

As an alternative to modifying the wild type gene after insertion into an expression vector, as described above with reference to plasmid pGEX-3X, genes encoding polypeptides of the invention may be made by methods which comprise the construction of vectors containing non-wild-type genes by ligation of oligonucleotides optionally followed by modification by, in particular, site-directed mutagenesis.

FIG. 2A of WO 98/42834 shows the nucleotide sequence of the synthetic dendroaspin (Den) gene. The gene was designed on the basis of the known amino acid sequence (Williams J. A et al ((1992)) Biochem Soc Trans 21: 73S) and the codons for each amino acid were adopted from those which were highly expressed in E coli (Fiers W ((1982)) Gene 18: 199-209) Ten synthetic oligonucleotides are shown in brackets and numbered individually 1 to 10 either above the coding strand or below the non-coding strand. The stop codon is indicated by an asterisk. Three-letter amino acid code is used and the total of 59 amino acids of Den are only numbered 1 for N-terminal residue arginine and 59 for C-terminal leucine.

In an additional aspect, therefore, the invention resides in nucleic acid molecules encoding a polypeptide of the invention. The nucleic acid may be operatively linked to a promoter and optionally to a nucleic acid sequence encoding a heterologous protein or peptide thereby to encode a fusion product. Suitably the promoter is IPTG inducible and optionally the heterologous protein or peptide is glutathione S-transferase.

Excluding the nucleic acid sequence encoding said further non-wild-type domain, nucleic acid sequences encoding the polypeptides of the invention may share about 50% nucleotide sequence homology, preferably about 65%, more preferably about 75% and even more preferably about 85% homology with a dendroaspin nucleotide sequence.

The invention includes plasmids comprising a nucleic acid of the invention, for example plasmid pGEX-3X comprising a nucleic acid of the invention, as well as host cells transformed with such a plasmid. A suitable host cell is E coli. The host cells may be provided as cell cultures.

Another aspect of the invention resides in a method of producing a polypeptide comprising culturing a host cell of the invention so as to express said polypeptide, extracting the polypeptide from the culture and purifying it.

The invention further includes a method of producing a polypeptide comprising a dendroaspin scaffold, the method comprising:

a) preparing an expression vector comprising a nucleic acid sequence encoding a dendroaspin scaffold of the invention operatively linked to a promoter and optionally linked to a nucleic acid sequence encoding a heterologous protein for co-expression therewith; and

b) transforming a host cell with the vector and causing the host cell to express the modified dendroaspin nucleic acid sequence.

Some of these methods comprise

a) (i) assembling from overlapping oligonucleotides the coding sequence of a dendroaspin scaffold containing an RGD motif and operatively linking the resulting cDNA to a promoter, the promoter optionally being linked to a nucleic acid sequence encoding a heterologous protein for expression of fusion protein; and

a) (ii) modifying the RGD-encoding domain of the expression vector to encode a dendroaspin scaffold in which RGD has been deleted or replaced by a replacement amino acid sequence as defined herein.

In such methods, step (a) (ii) may comprise, before or after said modification, modifying at least one other domain of the nucleic acid sequence of the vector encoding the dendroaspin scaffold by one or more of the insertion, deletion or substitution of nucleic acid residues so that on expression the dendroaspin scaffold comprises a corresponding domain having a non-wild-type dendroaspin sequence.

Others of the methods comprise constructing from oligonucleotides an expression vector comprising a nucleic acid sequence encoding a dendroaspin sequence in which the RGD-encoding domain has been deleted or replaced by a replacement amino acid sequence as defined herein and, optionally, modifying at least one other domain of the nucleic acid sequence of the vector encoding the dendroaspin scaffold by one or more of insertion, deletion or substitution of nucleic acid residues so that on expression the dendroaspin scaffold comprises a corresponding domain having a non-wild-type dendroaspin sequence.

The method may comprise the steps of:

a) extracting the modified dendroaspin from a host cell culture,

b) purifying the modified dendroaspin from the cell culture extract, and, if the modified dendroaspin is a fusion protein, cleaving the dendroaspin portion from the heterologous portion of the fusion protein.

The heterologous protein is suitably glutathione S-transferase (GST) and the purification suitably involves GST affinity chromatography followed by cleavage of the modified dendroaspin from GST.

Use

The peptides of the invention may be used for scientific investigations or, if pharmacologically active, may be used as pharmaceuticals.

We have found that the dendroaspin molecule provides an excellent scaffold for carrying “foreign” sequences and presenting them to potential targets. In this respect, the small size and conformational stability of the dendroaspin scaffold make it a good model for experimental use as well as pharmaceutical use. Moreover, the fact that the sequence and conformation of dendroaspin are known enables amino acid sequences to be inserted in a position where it can be predicted that they will be exposed.

A particular benefit of dendroaspin is that the RGD site is presented in a conformational environment which appears to improve association of the sequence at the RGD domain (RGD of course in wild type dendroaspin) with pockets in target structures as compared with linear peptides. Thus, the platelet-binding (GP IIb/IIIa receptor-binding) activity of RGD in dendroaspin is about 1,000 times greater than that of RGD linear peptide. The molecules of the invention are particularly useful, therefore, for presenting amino acid sequences to receptors and other structures having pockets.

Preferred polypeptides of the invention, therefore, have at the RGD domain a replacement amino acid sequence having receptor-binding activity. One class of polypeptides has at its RGD domain an amino acid sequence which, in its native polypeptide, enters a pocket to function.

The dendroaspin framework is useful for presenting amino acid sequences to targets for experimental purposes. Thus, the polypeptides of the invention are useful for investigating the function, effects or activity of “foreign” test sequences, e.g. for product development purposes. In other words, the polypeptides of the invention are useful for the purpose of developing active agents, especially for pharmaceutical purposes or to obtain information useful in the development of small molecule therapeutic or diagnostic agents, for example.

Accordingly, the present invention further provides a method for testing the biological, pharmacological and/or biochemical activity of a candidate amino acid sequence which method comprises incorporating the candidate sequence into a polypeptide according to the present invention. Preferably, the method further comprises exposing the test polypeptide thereby produced to a receptor, ‘pocket’ or other interactive entity (whether in vivo or in vitro) and, optionally, measuring the binding thereto or interaction therewith. Optionally, the test sequence may also be exposed to the receptor or other interactive entity in the presence of a control substance (whose response, e.g. binding or interaction, in the absence of the test polypeptide is known) and the response of the test polypeptide and/or of the control substance thereafter measured.

The present invention thereby provides a candidate amino acid sequence, e.g. polypeptide, identifiable by the test method according to the invention, its use as identifiable, and pharmaceutical formulations thereof. The invention further provides a molecule comprising such a candidate polypeptide, especially the test polypeptide incorporated in a dendroaspin scaffold as defined hereinbefore and (the residue of) the candidate polypeptide.

The pharmacologically active polypeptides may be formulated as a pharmaceutical composition comprising a polypeptide as hereinbefore defined, optionally further comprising a pharmaceutically acceptable excipient or carrier. A plurality of therapeutic polypeptides of the invention of different functionalities may be combined together in a pharmaceutically acceptable form so as to provide a desired treatment, and/or they may be combined with one or more other therapeutic or prophylactic agents.

The therapeutic polypeptides of the invention are preferably formulated for intravenous injection or intravenous infusion although other methods of administration are possible, e.g. oral, subcutaneous or intramuscular, should it be desired to provide a slow release into the circulatory system of an individual. Also possible is the formulation of the polypeptide for use with implanted controlled release devices such as those used to administer growth hormone, for example.

One formulation may comprise extravasated blood combined with a polypeptide of the invention at a concentration in the range 1 nM-60 μM. This blood may be stored in ready to use form and provides an immediate and convenient supply of blood for transfusion in cases when clotting must be avoided such as during or immediately following surgical procedures.

The invention includes a therapeutic polypeptide as hereinbefore defined for use in medicine, preferably as a pharmaceutical.

The invention also provides for the use of a pharmacologically active polypeptide as hereinbefore defined for the manufacture of a medicament, which may for example be for the treatment or prophylaxis of disease associated with binding at a receptor or with thrombosis; more particularly thrombosis, myocardial infarction, retinal neovascularization, endothelial injury, dysregulated apoptosis, abnormal cell migration, leukocyte recruitment, immune system activation, tissue fibrosis and tumorigenesis.

The invention also provides methods for the treatment by therapy or prophylaxis of diseases associated with binding at a receptor or with thrombosis; more particularly thrombosis, myocardial infarction, retinal neovascularization, endothelial injury, dysregulated apoptosis, abnormal cell migration, leukocyte recruitment, immune system activation, tissue fibrosis and tumorigenesis. The methods comprise administering a therapeutically effective amount of a polypeptide as hereinbefore defined.

### EXAMPLE 1

KGD-Dendroaspin (SEQ ID NO: 2)

Materials—Restriction enzymes, T4 polynucleotide kinase, T4 DNA ligase, IPTG (isopropyl-β-D-thio-galactopyranoside) and DH5α competent cells were purchased from Life Technologies Ltd (U.K.) or Promega Ltd. (Southampton, U.K.). Vent (exo-) DNA polymerase was supplied by New England Biolabs Ltd. (Hitchin, U.K.). Proteinase Factor Xa was purchased from Boehringer Mannheim (Sussex, England). Human fibrinogen (grade L) was purchased from Kabi (Stockholm, Sweden). Lyophilised snake venoms were obtained from either Latoxan (05150 Rosans, France) or Sigma Chemical Ltd (Dorset, U.K.). Oligonucleotides were made by Cruachem Ltd., (Glasgow, U.K.) and further purified by denaturing PAGE on a 15% acrylamide/8 M urea gel. Deoxynucleotide triphosphates (dNTPs), dideoxynucleotide triphosphates (ddNTPs) and plasmid pGEX-3X, a vector that expresses a cloned gene as a fusion protein linked to glutathione S-transferase (GST), and Glutathione-Sepharose CL-4B were purchased from Pharmacia Biotech Ltd. (Herts, U.K.). “Geneclean” kit and Plasmid maxi Kit were purchased from Bio 101, La Jolla Calif. U.S.A. and Qiagen Ltd., Surrey, U.K. respectively. The sequencing enzyme (Sequenase 2.0) was obtained from Cambridge Bioscience (Cambridge, U.K.). [³⁵S]dATP[αS] and ¹²⁵I (15.3 mCi/mg iodine) were supplied by NEN Dupont (Herts, U.K.) and Amersham International Plc (Amersham, Bucks, England), respectively.

Construction of the expression vectors—A dendroaspin gene was constructed from synthetic oligonucleotides, using the same 10 oligonucleotides shown in FIG. 2A of WO 98/42834. Each purified oligonucleotide was phosphorylated at 37° C. for 60 min in the presence of 1 mM ATP and T4 polynucleotide kinase. Each pair of overlapping phosphorylated oligonucleotides was annealed separately on a Perkin-Elmer/Cetus thermal cycler. The following programme was used: 95° C. 5 min, 70° C. 30s then slowly cooling to room temperature. Ligation was performed at 16° C. for 15 hours in a total volume of 50 μl containing approx. 1 nM of each annealed fragment, 50 mM Tris-HCl (pH 7.6), 10 mM MgCl₂, 1 mM ATP and 5% PEG 8000 and 5 units of T4 DNA ligase. After ligation, the dendroaspin gene was amplified by PCR using 1 μl of ligation mixture as template with oligo 1 and 10 as primers and 2 units of Vent DNA polymerase. The following programme was applied: one cycle of 3 min at 94° C. and 1 min at 72° C., followed by 39 cycles of 30s at 94° C., and 2 min at 72° C. The amplification product was checked and found to be of expected size (216 bp) as ascertained on a 2% agarose gel and further purified on a 2% low-melting-point agarose gel. The dendroaspin gene was digested with EcoRI and BamHI and then cloned into the restriction vector pGEX-3X to produce recombinant plasmid pGEX-Dendroaspin gene. The same protocol is followed in the construction of the non-wild type expression vectors, for example the pGEX-KGD-Dendroaspin gene (see below).

The KGD-dendroaspin gene was produced by using a Transformer site-directed mutagenesis kit (Clontech Laboratories Inc, Palo Alto, Calif., USA). A selection oligonucleotide was designed to introduce a novel restriction site (BamHI→ACC65I) into the PGEX-3X vector to allow selection of recombinant from parental constructs by digestion with ACC65I. After annealing, ligation and digestion, the reaction mixture was transformed into E coli mut S cells (Clontech) and subsequent colonies were screened by ACC65I restriction analysis. After two or three rounds of restriction with ACC65I and transformation, more than 90% recombinant clones were identified. In the mutagenesis procedure, there were used the selection primer dGAAGGTCGTGGGTACCATATCGAAGGTCGT (SEQ ID NO: 17) and the mutagenesis primer dTGCTTCACTCCGAAAGGTGACATGCCGGGTCCGTAC (SEQ ID NO: 18).

Transformation and protein expression—Recombinant gene (5 ng) was used to transform 50 μl of E. coli DH5α competent cells by standard methods (Sambrook et al. (1989) Molecular Cloning: A Laboratory Manual, Cold Spring Harbor Laboratory Press, Cold Spring Harbor, N.Y.). The presence of correct coding sequence of the constructs was verified by complete DNA sequencing of the inserted fragments using the dideoxy chain-termination method. (Sanger et al. (1977) Proc. Natl. Acad. Sci. U.S.A 74, 5463-5467. Bacterial culture conditions were carried out as follows: the culture was inoculated with an overnight seed culture (1%, v/v) and grown in LB/ampicillin medium (100 μg/ml) and shaken at 37° C. until it reached an A₆₀₀ of 0.7, then IPTG was added to a final concentration of 0.1 mM for induction. The cells were grown for an additional 4 hours at a lower temperature of 30° C. and harvested by centrifugation.

Purification of native and recombinant snake venom RGD proteins—Elegantin, and dendroaspin were purified using reverse-phase HPLC as described previously (Lu et al. (1993) Biochem. J. 296, 21-24). Recombinant dendroaspins were purified as follows: the cell pellets were suspended in PBS buffer (pH 7.4) containing 1% Triton X-100 and the protease inhibitors PMSF (1 M), pepstatin (5 g/ml), aprotinin (5 μg/ml), trypsin inhibitor (1 μg/ml), 1 mM EDTA, and sonicated on ice. The sonicated mixture was centrifuged at 7,800×g at 4° C. to pellet the cell debris and insoluble material. Recombinant GST-dendroaspin and GST-mutant-dendroaspins from supernatants were purified by affinity chromatography on glutathione-Sepharose CL-4B columns by absorption in PBS containing 150 mM NaCl and elution with 50 mM Tris-HCI containing 10 mM reduced glutathione (pH 8.0). With the remaining insoluble fusion protein in the pellets, solubilisation was achieved in the presence of 8 M urea, by gently shaking at room temperature for 30 min and subsequent renaturation by continual dilution and dialysis at room temperature against Tris-HCI buffer. The refolded fusion protein mixture was subjected to further centrifugation and affinity-purification. The purification was monitored by SDS-PAGE and the appropriate fractions comprising the recombinant GST-Dendroaspin and GST-mutant-dendroaspins were digested in the presence of 150 mM NaCl, 1 mM CaCl₂ and Factor Xa (1:100, w/w Factor Xa:fusion protein) at 4° C. for 24 hours. After cleavage, the fractions were loaded onto a Vydac C₁₈ reverse phase HPLC analytical column (TP104) and eluted with a gradient of 0-26% acetonitrile (1.78% per min) containing 0.1% trifluoroacetic acid (TFA), followed by 26-36% acetonitrile in 0.1% TFA (0.25% per min). When necessary, further analytical columns were run under the same conditions. The fractions from HPLC were freeze-dried, dissolved in water and assayed for the inhibition of ADP-induced platelet aggregation. Purified wild-type dendroaspin and mutants were characterised by 20% SDS-PAGE and electrospray ionisation mass spectrometry.

Measurement of platelet aggregation—Platelet aggregation was measured by the increase in light transmission as described previously (Lu et al. (1993), supra; Lu et al. (1994) Biochem. J. 304, 929-936). Briefly, platelet rich plasma (PRP) was prepared from citrated human blood, obtained from healthy individuals, by centrifugation at 200×g for 15 min. Washed platelets were prepared from PRP and resuspended in adhesion/aggregation buffer (145 mM NaCl, 5 mM KCl, 1 mM MgCl₂, 2 mM CaCl₂, 10 mM glucose, 3.5 mg/ml BSA and 10 mM HEPES, pH 7.35) and adjusted to a count of 3×10⁸/ml. Platelet aggregation (320 μl incubations) was induced with 10 μM ADP in the presence of 1.67 mg/ml fibrinogen and measured using a Payton Dual-Aggregometer linked to a chart recorder. KGD-dendroaspin (SEQ ID NO: 2) was found to show potent inhibition of ADP-induced platelet aggregation.

Measurement of platelet adhesion—Platelet adhesion is measured as described previously (Lu et al. (1994), supra). Briefly, 96 well plates are coated overnight at 4° C. with either human fibrinogen or fibronectin reconstituted in phosphate buffered saline (PBS) (pH 7.4) at appropriate concentrations (2-10 μg/ml, 100 μl). Platelets are treated with antagonists at appropriate concentrations for 3 min before the addition (90 μl) to the microtitre plates which are pre-loaded with 10 μl of 500 μM ADP (final conc. 50 μM) and the number of adherent platelets is determined by measurement of endogenous acid phosphatase using 130 μl/well of the developing buffer (sodium acetate, pH 5.5, 10 mM p-nitrophenyl phosphate, 0.1% Triton X-100) and read at 410/630 nm on an automated plate reader.

Iodination of Ligands and Ligand Binding Studies—Iodination of all proteins used in this study is performed using Enzymobead Radioiodination Reagent (Biorad Laboratories) according to the manufacturer's specifications. The binding of ¹²⁵I-labelled disintegrins, dendroaspin and mutant dendroaspins to washed platelets is performed under equilibrium conditions essentially as described previously (Lu et al. (1994), supra). Briefly, the incubation mixture is composed of 300 μl of washed platelets (3×10⁸/ml), 10 μl of agonist (1.75 mM ADP giving a final conc. of 50 μM), 10 μl of ¹²⁵I-labelled protein samples, 5-20 μl resuspension buffer and made to a final volume of 350 μl. In antibody inhibition studies, platelet suspensions are treated with antibody for 30 min prior to exposure to ADP and then added to ¹²⁵I-protein samples and the mixture incubated at room temperature for a farther 60 min. Incubations are terminated by loading onto a 25% (w/v) sucrose, 1% BSA cushion and centrifugation at 12,000×g for 10 min. Both platelet pellets and supernatants are counted to determine the levels of bound and free ligand. Background binding levels are determined in the presence of a 50-fold excess of cold disintegrin or 10 mM EDTA.

Expression and purification of recombinant wild-type dendroaspin and mutant-dendroaspins—The synthetic wild-type and mutated dendroaspin genes were cloned into the expression vector pGEX-3X at the carboxyl terminus of the glutathione S-transferase (GST) gene with a Factor Xa cleavage sequence positioned 5′ of the gene coding for these recombinant proteins. The expression of the GST-fusion protein in E. coli was induced by addition of IPTG to the growth medium, as described under the headings “Construction of the expression vector” and “Transformation and protein expression”. In contrast to non-induced transformants, analysis of IPTG treated cell lysates by SDS-PAGE showed an emergence of a 32 kDa protein corresponding to the GST-fusion protein. The GST-protein was purified by affinity chromatography on glutathione-Sepharose CL-4B column and monitored by SDS-PAGE. Elution of the absorbed material with glutathione resulted in the appearance of a major band migrating at 32 kDa and a minor band at 28 kDa in 12.5% polyacrylamide gels. This minor 28 kDa component may correspond to free GST released from the GST-protein by an endogenous bacterial protease with Factor Xa-like activity since the relative levels of this species varied with different preparations. Treatment of the purified GST-proteins with Factor Xa released recombinant proteins migrating as 7 kDa bands, approximating the size of dendroaspin, and free GST appearing as an intensification of the 28 kDa band identified by SDS-PAGE. The 7 kDa protein was further purified to homogeneity by reverse-phase HPLC with the active fraction identified by testing aliquots from each peak for their ability to inhibit ADP-induced platelet aggregation in PRP. Further characterisation by mass spectrometry confirmed the successful cleavage at Arg¹ by Factor Xa protease treatment.

Modified Molecules—FIG. 1 (SEQ ID NOS: 1-14) shows the sequences of modified monofunctional and bifunctional dendroaspins obtainable by mutagenesis of the dendroaspin gene as described in the specification and in WO 98/42834. The sequences of these molecules are shown in the sequence listing.

### EXAMPLE 2

KQAGDV-Dendroaspin (SEQ ID NO: 8)

The same procedures as described in Example 1 were followed to express and purify KQAGDV-dendroaspin (SEQ ID NO: 8). The mutagenesis primer used in the site-directed mutagenesis was:

dGGT TGC TTC ACT CCG AAA CAG GCT GGT GAC GTT CCG GGT CCG TACO TGC (SEQ ID NO: 19),

corresponding to the amino acid sequence:

GCFTPKQAGDVPGPYC (SEQ ID NO: 20).

It will be appreciated from the aforegoing that the invention provides the use of dendroaspin as a scaffold for one or more non-dendroaspin amino acid sequences in a dendroaspin framework in which the native RGD motif has been deleted or has been replaced by (i) an amino acid sequence having no integrin-binding activity or (ii) an aspartic acid- or glutamic acid-containing integrin-binding amino acid sequence other than RGD.

1. A polypeptide comprising a dendroaspin scaffold in which the native RGD motif has been deleted or has been replaced by a replacement amino acid sequence which is (i) an amino acid sequence having no integrin-binding activity or (ii) an integrin-binding amino acid sequence and comprising a tripeptide sequence other than RGD containing D or E adjacent to G.

2. A polypeptide of claim 1 in which the tripeptide sequence is of the formula
B-J-Z
wherein
I) J-Z is GD or GE and Bis R, K, Q, A, H, N, A, V, I, L, M, F, P or W;
II) B-J is DG or EG and Z is any amino acid; or
iii) J is D or E and B and Z are each independently selected from A, V, I, L, M, F, P or W.

3. A polypeptide of claim 2(I) in which J-Z is GD.

4. A polypeptide of claim 2(I) or claim 4 in which B is R, K, Q, A, H or N, provided that B-J-Z is not RGD.

5. A polypeptide of claim 4 in which B is R, K, Q or A.

6. A polypeptide of claim 2(I) in which B-J-Z is bonded at its C-terminal end to M, W, N or V.

7. A polypeptide of claim 6 in which said M, W, N or V residue is followed by the P which is at position 47 of wild type dendroaspin or by an A residue substituted therefor.

8. A polypeptide of 2(I) in which the integrin-binding amino acid sequence is preceded by the P which is at position 42 of wild type dendroaspin or by an A residue substituted therefor.

9. A polypeptide of claim 2(I) in which B is A, V, I, M, F, P, W.

10. A polypeptide of claim 9 in which B is L or V.

11. A polypeptide of claim 10 in which B is L and is preceded by M.

12. A polypeptide of claim 2(II) in which B-J is DG.

13. A polypeptide of claim 2(II) in which Z is E. R or P.

14. A polypeptide of claim 2(II) in which Z is followed by the P which is at position 47 of wild type dendroaspin or by an A inserted before the wild type position 47 P.

15. A polypeptide of claim 2(III) in which J is D.

16. A polypeptide of claim 2(III) in which B-J-Z is LDV.

17. A polypeptide of claim 2(III) in which B-J-Z is preceded by an I residue.

18. A polypeptide of claim 1 which comprises a said replacement amino acid sequence (i) having no integrin binding activity, the replacement amino acid sequence having a receptor-binding function.

19. A polypeptide of claim 1 which comprises a said replacement amino acid sequence (i) having no integrin binding activity, the replacement amino acid sequence being one which in its native polypeptide enters a pocket to function.

20. A polypeptide of claim 1 which, in addition to deletion or replacement of the RGD motif, comprises at least one non-wild-type dendroaspin domain.

21. A polypeptide of claim 20, wherein the at least one non-wild-type dendroaspin domain comprises at least one non-dendroaspin sequence which confers functionality on the polypeptide.

22. A polypeptide of claim 20, comprising at least two said non-wild-type dendroaspin domains, the non-wild-type dendroaspin domains optionally having the same sequence.

23. A polypeptide of claim 20 wherein said at least one non-wild-type dendroaspin domain comprises a said domain having two or more amino acid sequence portions separated by at least one amino acid residue of dendroaspin.

24. A polypeptide of claim 20 which contains a said non-wild-type dendroaspin domain conferring platelet derived growth factor (PDGF) activity, glycoprotein IBα activity, hirudin activity, thrombomodulin activity, vascular epidermal growth factor activity, transforming growth factor-β1 activity, basic fibroblast growth factor activity, angiotensin II activity, factor VIII activity, von Willebrand factor activity, tick anticoagulant protein (TAP) activity or nematode anticoagulant protein (NAP) activity.

25. A polypeptide of claim 24, wherein the non-wild-type dendroaspin domain is a sequence derived from platelet derived growth factor (PDGF), glycoprotein IBα, hirudin, thrombomodulin, vascular epidermal growth factor, transforming growth factor-β1, basic fibroblast growth factor, angiotensin II, factor VIII, von Willebrand factor, tick anticoagulant protein (TAP) or nematode anticoagulant protein (NAP), or a sequence having homology to at least part of such sequence.

26. A polypeptide of claim 20, wherein the non-wild-type domains is/are incorporated into (a) loop I and/or loop II; (b) loop I and/or loop III; (c) loop II and/or loop III; or loop I, loop II and loop III of the dendroaspin scaffold.

27. A polypeptide of claim 26, wherein there is a single said non-wild-type domain and the domain is incorporated into either loop I or loop II.

28. A polypeptide of claim 20, wherein the non-wild-type domains is/are contained in the dendroaspin scaffold between amino acid residues selected from one or more of 4-16, 18-21 or 23-36, or at the end of the dendroaspin scaffold after residue 50.

29. A polypeptide of claim 28, in which there is a single non-wild-type domain.

30. A polypeptide of claim 1, wherein loop III is additionally modified as compared with native dendroaspin by insertion, deletion or substitution of one or more amino acid residues

31. A polypeptide of claim 30, wherein a maximum of 8 and a minimum of 1 amino acids are modified by said additional modification within loop III.

32. A polypeptide of claim 30, wherein RGD has been replaced by a said integrin binding sequence and said additional modification comprises modification of the amino acids flanking said integrin binding sequence.

33. A polypeptide of claim 1, wherein loop I and/or loop II are additionally modified by insertion, deletion or substitution of one or more amino acid residues.

34. A polypeptide of claim 1, which contains not more than 36 amino acid residues more than native dendroaspin.

35. A polypeptide of claim 34, which contains from 14 to 36 amino acid residues more than native dendroaspin.

36. A nucleic acid molecule encoding a polypeptide of claim 20.

37. A nucleic acid of claim 36, operatively linked to a promoter and optionally to a nucleic acid sequence encoding a heterologous protein or peptide thereby to encode a fusion product.

38. A nucleic acid of claim 37, wherein the promoter is IPTG inducible and optionally the heterologous protein or peptide is glutathione S-transferase.

39. A plasmid comprising a nucleic acid of claim 36.

40. Plasmid pGEX-3X comprising a nucleic acid of claim 36.

41. A host cell transformed with a plasmid of claim 39.

42. A host cell of claim 41 which is E coli.

43. A cell culture comprising host cells of claim 41.

44. A method of producing a polypeptide as defined in claim 20 comprising culturing a host cell of claim 41 so as to express said polypeptide, extracting the polypeptide from the culture and purifying it.

45. A method of producing a polypeptide comprising a dendroaspin scaffold, the method comprising:
a) preparing an expression vector comprising a nucleic acid sequence encoding a dendroaspin scaffold of claim 1 operatively linked to a promoter and optionally linked to a nucleic acid sequence encoding a heterologous protein for co-expression therewith; and
b) transforming a host cell with the vector and causing the host cell to express the modified dendroaspin nucleic acid sequence.

46. A method of claim 45, wherein step (a) comprises
a) (i) assembling from overlapping oligonucleotides the coding sequence of a dendroaspin scaffold containing an RGD motif and operatively linking the resulting cDNA to a promoter, the promoter optionally being linked to a nucleic acid sequence encoding a heterologous protein for expression of fusion protein; and
a) (ii) modifying the RGD-encoding domain of the expression vector to encode a dendroaspin scaffold in which RGD has been deleted or replaced by a replacement amino acid sequence as defined in claim 1.

47. A method of claim 46, in which step (a) (ii) further comprises, before or after said modification, modifying at least one other domain of the nucleic acid sequence of the vector encoding the dendroaspin scaffold by one or more of the insertion, deletion or substitution of nucleic acid residues so that on expression the dendroaspin scaffold comprises a corresponding domain having a non-wild-type dendroaspin sequence.

48. A method of claim 45, wherein step (a) comprises constructing from oligonucleotides an expression vector comprising a nucleic acid sequence encoding a dendroaspin sequence in which the RGD-encoding domain has been deleted or replaced by a replacement amino acid sequence as defined in claim 1 and, optionally, modifying at least one other domain of the nucleic acid sequence of the vector encoding the dendroaspin scaffold by one or more of insertion, deletion or substitution of nucleic acid residues so that on expression the dendroaspin scaffold comprises a corresponding domain having a non-wild-type dendroaspin sequence.

49. A method of claim 44, further comprising the steps of:
d) extracting the modified dendroaspin from a host cell culture,
e) purifying the modified dendroaspin from the cell culture extract, and, if the modified dendroaspin is a fusion protein, cleaving the dendroaspin portion from the heterologous portion of the fusion protein.

50. A method of claim 49 wherein the heterologous protein is glutathione S-transferase (GST) and the purification involves GST affinity chromatography followed by cleavage of the modified dendroaspin from GST.

51. A polypeptide of claim 1 having the characteristics of a polypeptide obtained by the method of claim 44.

52. A pharmaceutical composition comprising a pharmacologically active polypeptide of claim 22.

53. A composition as claimed in claim 52, further comprising a pharmaceutically acceptable excipient or carrier.

54. A method for the treatment or prophylaxis of a disease associated with thrombosis in a human or animal patient, comprising administering to the patient an effective amount of a polypeptide as claimed in claim 20.

55. A method of investigating the function, effects or activity of a non-wild-type dendroaspin sequence, comprising providing a polypeptide of claim 1 which comprises said sequence and performing in vivo or in vitro tests with said polypeptide.

56. A pharmaceutical formulation, comprising an amino acid sequence whose functions, effects or activity were investigated by carrying out the method of claim 55.


SEQUENCE LISTING (21 sequences)

SEQ ID NO: 1 - protein, 59 aa, Dendroaspis jamesoni
RICYNHLGTKPPTTETCQEDSCYKNIWTFDNIIRRGCGCFTPRGDMPGPYCCESDKCNL

SEQ ID NO: 2 - protein, 59 aa, Artificial
  Modified dendroaspin (KGD-dendroaspin)
RICYNHLGTKPPTTETCQEDSCYKNIWTFDNIIRRGCGCFTPKGDMPGPYCCESDKCNL

SEQ ID NO: 3 - protein, 59 aa, Artificial
  Modified dendroaspin (KQA-dendroaspin)
RICYNHLGTKPPTTETCQEDSCYKNIWTFDNIIRRGCGCFTPKQAMPGPYCCESDKCNL

SEQ ID NO: 4 - protein, 59 aa, Artificial
  Modified dendroaspin (KGDW-dendroaspin)
RICYNHLGTKPPTTETCQEDSCYKNIWTFDNIIRRGCGCFTPKGDWPGPYCCESDKCNL

SEQ ID NO: 5 - protein, 59 aa, Artificial
  Modified dendroaspin (QGDM-dendroaspin)
RICYNHLGTKPPTTETCQEDSCYKNIWTFDNIIRRGCGCFTPQGDMPGPYCCESDKCNL

SEQ ID NO: 6 - protein, 59 aa, Artificial
  Modified dendroaspin (AGDV-dendroaspin)
RICYNHLGTKPPTTETCQEDSCYKNIWTFDNIIRRGCGCFTPAGDVPGPYCCESDKCNL

SEQ ID NO: 7 - protein, 59 aa, Artificial
  Modified dendroaspin (AGDM-dendroaspin)
RICYNHLGTKPPTTETCQEDSCYKNIWTFDNIIRRGCGCFTPAGDMPGPYCCESDKCNL

SEQ ID NO: 8 - protein, 61 aa, Artificial
  Modified dendroaspin (KQAGDV-dendroaspin)
RICYNHLGTKPPTTETCQEDSCYKNIWTFDNIIRRGCGCFTPKQAGDVPGPYCCESDKCNL

SEQ ID NO: 9 - protein, 59 aa, Artificial
  Modified dendroaspin (QGDV-dendroaspin)
RICYNHLGTKPPTTETCQEDSCYKNIWTFDNIIRRGCGCFTPQGDVPGPYCCESDKCNL

SEQ ID NO: 10 - protein, 59 aa, Artificial
  Modified dendroaspin (DGEA-dendroaspin)
RICYNHLGTKPPTTETCQEDSCYKNIWTFDNIIRRGCGCFTPDGEAPGPYCCESDKCNL

SEQ ID NO: 11 - protein, 59 aa, Artificial
  Modified dendroaspin (DGPA-dendroaspin)
RICYNHLGTKPPTTETCQEDSCYKNIWTFDNIIRRGCGCFTPDGPAPGPYCCESDKCNL

SEQ ID NO: 12 - protein, 73 aa, Artificial
  Modified dendroaspin (KGDM-dendroaspin-Hr)
RICFTPRGDMPGPYPGPCQEDSCYKNIWTFDNIIRRGCGCFTPKGDMPGPYCFPRPQSHNDGDFEEIPEEYLQ

SEQ ID NO: 13 - protein, 70 aa, Artificial
  Modified dendroaspin (QGDV-dendroaspin-Tm)
RICFTPRGDMPGPYPGPCQEDSCYKNIWTFDNIIRRGCGCFTPQGDVPGPYECPEGYILDDGFICTDIDE

SEQ ID NO: 14 - protein, 66 aa, Artificial
  Modified dendroaspin (KGDW-dendroaspin-Gb)
RICFTPRGDMPGPYPGPCQEDSCYKNIWTFDNIIRRGCGCFTPKGDWPGPYGDTDLYDYYPEEDTE

SEQ ID NO: 15 - protein, 6 aa, Artificial
  inhibitory synthetic peptide analogue
KQAGDV

SEQ ID NO: 16 - protein, 4 aa, Artificial
  thrombin-binding sequence
GPRP

SEQ ID NO: 17 - DNA, 30 bp, Artificial
  selection primer
gaaggtcgtgggtaccatatcgaaggtcgt

SEQ ID NO: 18 - DNA, 36 bp, Artificial
  mutagenesis primer
tgcttcactccgaaaggtgacatgccgggtccgtac

SEQ ID NO: 19 - DNA, 48 bp, Artificial
  mutagenesis primer
ggttgcttcactccgaaacaggctggtgacgttccgggtccgtactgc

SEQ ID NO: 20 - protein, 16 aa, Artificial
GCFTPKQAGDVPGPYC

SEQ ID NO: 21 - protein, 58 aa, Artificial
  Modified dendroaspin (LDV-dendroaspin)
RICYNHLGTKPPTTETCQEDSCYKNIWTFDNIIRRGCGCFTPLDVPGPYCCESDKCNL